(19) United States
(12) Patent Application Publication
(10) Pub. No.: US 2012/0322985 A1
(43) Pub. Date: Dec. 20, 2012

(54) FOXO6 POLYCLONAL ANTIBODY AND METHOD FOR PREPARING THE SAME

(75) Inventors: SHEN-LIANG CHEN, Taoyuan County (TW); CHING-WEN SU, New Taipei City (TW)

(73) Assignee: NATIONAL CENTRAL UNIVERSITY, Jhongli City (TW)

(21) Appl. No.: 13/242,280

(22) Filed: Sep. 23, 2011

(30) Foreign Application Priority Data
Jun. 14, 2011 (TW) 100120777

(51) Int. Cl. C07K 16/18; C12P 21/00; C07K 1/00

(52) U.S. Cl. 530/387.9; 435/71.1

(57) ABSTRACT

A FoxO6 polyclonal antibody is provided. The FoxO6 polyclonal antibody recognizes a FoxO6 sequence having a fragment SEQ ID NO:1 without recognizing any sequence selected from a group consisting of a FoxO1, a FoxO3, a FoxO4, and a combination thereof.

### CROSS-REFERENCE TO RELATED APPLICATION AND CLAIM OF PRIORITY

This application claims the benefit of Taiwan Patent Application No. 100120777, filed on Jun. 14, 2011, in the Taiwan Patent and Trademark Office, the disclosures of which are incorporated herein in their entirety by reference.

### FIELD OF THE INVENTION

The present invention relates to a FoxO6 polyclonal antibody with high specificity and the method for preparing the same.

### BACKGROUND OF THE INVENTION

Forhead box (FOX) is a transcription factor related to cell physiology, wherein the subclass of FoxO includes FoxO1, FoxO3, FoxO4, and FoxO6 which are all related to the process of the cell physiology, such as regulation of the cell cycle, DNA repair, cell differentiation, and apoptosis.

With regard to the FoxO6 transcription factor, it is recently found and its role in the process of the cell physiology and expression pattern are not fully established. In order to study the FoxO6 transcription factor, there are commercially available antibodies for experiment research. Currently, the commercial FoxO6 polyclonal antibody sold by the Abcam company is produced by synthesizing peptide corresponding to a region within amino acids 181-230 of mouse FoxO6 (mFoxO6) as an antigen, and injecting it into a rabbit to generate a FoxO6 polyclonal antibody. However, the specificity of the FoxO6 polyclonal antibody sold by the Abcam company is not satisfying to researchers. These drawbacks lead to inconvenience for research.

In order to overcome the drawbacks in the prior art, a FoxO6 polyclonal antibody and a method for preparing the same product are provided in the present invention. The particular design in the present invention not only solves the problems described above, but also is easy to be implemented. Thus, the present invention has the utility for the industry.

### SUMMARY OF THE INVENTION

In accordance with an aspect of the present invention, a FoxO6 polyclonal antibody is provided. The FoxO6 polyclonal antibody recognizes a FoxO6 sequence having a fragment SEQ ID NO:2 without recognizing a sequence being one selected from a group consisting of a FoxO1, a FoxO3, a FoxO4, and a combination thereof.

In accordance with another aspect of the present invention, a method for preparing a FoxO6 polyclonal antibody is provided. The method includes steps of purifying a FoxO6 sequence having a fragment SEQ ID NO:2; mixing the FoxO6 sequence having the fragment SEQ ID NO:2 with an adjuvant to form an antigen; and injecting the antigen into a mammal to generate the FoxO6 polyclonal antibody.

In accordance with a further aspect of the present invention, a method for preparing a FoxO6 polyclonal antibody is provided. The method includes steps of providing a FoxO6 sequence having a fragment SEQ ID NO:2 as an antigen; and injecting the antigen into a mammal to generate the FoxO6 polyclonal antibody.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 shows a result of the antibody of the present invention recognizing mouse or human FoxO6;

FIGS. 2a and 2b show the specificity of the antibody of the present invention for the FoxO family proteins generated by in vitro transcription and translation;

FIG. 3a shows a result of the antibody of the present invention recognizing FoxO6 of cell total lysate origin; and

FIG. 3b shows a result of the antibody of the commercial product (Abcam) recognizing FoxO6 of cell total lysate origin;

FIG. 4a shows an antigen-antibody affinity result of the commercial product with increasing dose of pure MBP-FoxO6 protein; and

FIG. 4b shows an antigen-antibody affinity result of the present invention with increasing dose of pure MBP-FoxO6 protein.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

Determination of the FoxO6 Antigen Sequence

By comparing the coding region of the mouse FoxO family (mFoxO1, mFoxO3, mFoxO4 and mFoxO6) and their protein sequences (the nucleotide sequence and protein sequence information could be obtained from NCBI website), it is found that the region between nucleotides 685-1476 of mouse Foxo6 possess large diversity compared to other FoxOs. Accordingly, the region between amino acids 229-492 is chosen for antigen expression.

Construction of the Expression Vector pET-32a-mut-mFoxO6 +685˜+1476

1. Construction of the Vector DNA (pET-32a-mut)

The pET-32a (commercially available, Novagen) is cut by restriction enzyme Not I in 37 for 7 hours, and then the Xho I is added in 37 for overnight. The 1 μl 10 mM dNTP, 1 μl Klenow polymerase are added in 37 for 1 hour. The 1 μl 0.5M pH 8.0 EDTA is added and put in 75 for 10 minutes to terminate enzyme reaction. The pET-32a-mut is generated by self-ligation reaction of the purified DNA vector. In order to confirm that the mutant vector is correctly produced, the clones where Not I and Xho I cutting sites are destroyed are selected by the transformation and restriction cutting method. After BamHI and EcoRI are reacted, the Calf Intestinal alkaline Phosphatase (CIP, NEB, U.S.A.) is added to remove 5′ phosphate of the vector.

2. Construction of the Insert DNA (mFoxO6+685˜+1476)
(1) Construction of the pEGFP-N1-FoxO6: The total RNA is extracted from the adult mice brain, and then the FoxO6 sequence produced by RT-PCR is cloned into pEGFP-N1 (commercially available) by cutting sites Sal I and Bam HI.
(2) Construction of pcDNA3.0-mFoxO6 CDS full length: The pEGFP-N1-FoxO6 is treated by HindIII and BamHI in 37° C. incubator for overnight. The DNA (mFoxO6 CDS full length) which is completely cut is confirmed by analyzing the 0.8% agarose gel electrophoresis. Then the DNA is purified from gels.
(3) The 792 b.p. fragments are amplified by PCR with mixture including DMSO, pcDNA3.0-mFoxO6 CDS full length as template, forward primer and reverse primer. The purified PCR products are treated with EcoRI and BamHI in 37 incubator for overnight.

3. Ligation

The ligation is reacted on the 5′ phosphate removed vector DNA and the insert DNA in 25 for 16 hours, and then they are heated in 75 for minutes. The products (pET-32a-mut-mFoxO6+685˜+1476) are subsequently used for E. coli transformation and induction mFoxO6 antigen protein expression.

Induction mFoxO6 Antigen Protein Expression

The constructed pET-32a-mut-mFoxO6+685˜+1476 plasmid is transformed into E. coli competent cell (BL 21) at 37 and incubated for 12˜16 hours. A selected single colony is incubated in 3 ml LB incubation medium with 50 μg/ml Ampicillin in 37 incubation for 12˜16 hours. The bacteria are amplified by shaking with 1:100 dilution inoculation in 37 incubation for 3.5˜4 hours. When reaching O.D value about 0.7, the final concentration of 0.1 mM IPTG is added and then incubated in 37 incubator for 3˜3.5 hours. The bacteria is pelleted by centrifuging at 6,000 rpm, 4 for 10 minutes are re-suspended by 30 ml 1×binding buffer with 1 μM Leupeptin, 1 μM Aprotinin and 1 mM PMSF, and lysed by the French press method. The protein supernatants are obtained by centrifuging at 13,000 rpm, 4 for 1 hour, and stored at −20 for ready-for-use.

Purification of the mFoxO6 Antigen Proteins

The mFoxO6 in above-mentioned protein supernatants is purified with commercial kit Chelating Sepharose™ Fast Flow (Amersham Pharmacia Biotech AB, catalog #17-0575-01). The purified proteins are frozen at −20 ready for use. In addition to inducing mFoxO6 antigen protein expression in microorganisms, the mFoxO6 antigen proteins could also be synthesized by a machine.

Injection of the mFoxO6 Antigen

In the first week of immunization, the 200 μg antigen proteins are diluted to about 1 ml with PBS. First, the equal volume of Freund's complete adjuvant (CFA) and diluted antigen proteins are well mixed. After the emulsification between adjuvant and the antigen is well mixed, the 500 mg/ml urethane is injected into 8˜10 weeks aged New Zealand White Rabbit. The dosage for every time is about 1000 mg/kg. The antigens are separately injected into the subcutaneous tissue of the rabbits' back. About 100 μl antigens are injected into every injection point. From the second to the ninth week of immunization, the 100 μg antigens are injected every time, and the mixture of the equal volume of Freund's incomplete adjuvant (IFA) and diluted antigen proteins are injected after emulsification by mixing.

Collection of the Rabbit Serum/Plasma

Before immunization of the mouse FoxO6 antigen: The rabbit is fastened, and then the needle is rinsed with 0.3% heparin. The blood collected from the blood capillary of the back ear of the rabbit is centrifuged at 3,000 rpm, 4 to separate red blood cells from plasma. The separated plasma is frozen at −20.

After immunization of the mFoxO6 antigen: The blood is collected from the rabbit heart by syringes. After the blood is coagulated, the blood collection tubes are centrifuged in the centrifuge at 3,000 rpm, 4 for 5 minutes. The supernatants (serums) are separately transferred into eppendorfs, and 4-(2-hydroxyethyl)-1-piperazineethanesulfonic acid (HEPES) buffers are added to store serums and frozen at −80 ready for use.

Determination of the Recognizing Pattern of the FoxO6 Polyclonal Antibody for Human FoxO6

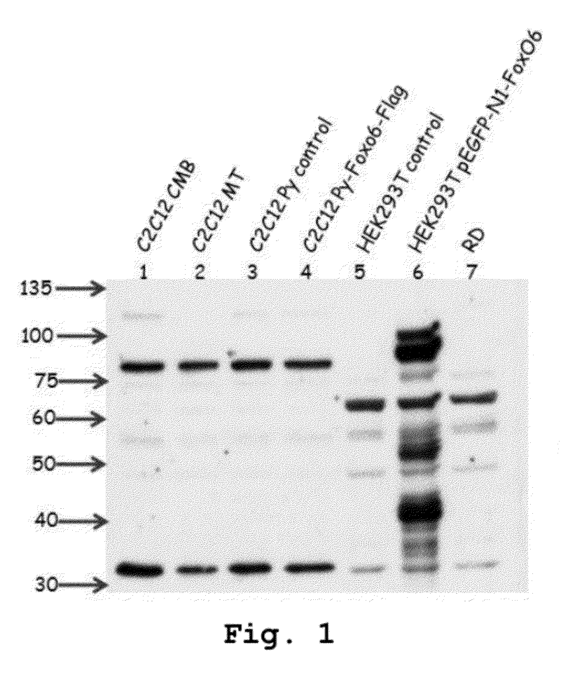

The Western blot is performed with the FoxO6 polyclonal antibody (rabbit serum) as the primary antibody (1:10⁴ dilution) and anti-rabbit-HRP as the secondary antibody (1:10⁴ dilution) to detect the following groups: CMB (confluent) stage myoblast (C2C12 CMB), myotube stage myoblast (C2C12 MT), vector-stable-expressed myoblast (C2C12 Py-vector control), FoxO6-Flag-stable-expressed myoblast (C2C12 Py-FoxO6-Flag), non-transfected human embryonic kidney control (HEK control), mFoxO6-transfected overexpressed HEK 293T (HEK FoxO6-GFP) and rhabdomyosarcoma (RD). Lanes 4˜5 of FIG. 1 show that the antibody of the present invention not only recognizes mouse FoxO6 but also recognizes human FoxO6 (the molecular weight of the human FoxO6 is smaller than that of the mouse FoxO6).

TNT in Vitro Translation of FoxO1, FoxO3, FoxO4 and FoxO6 Lysates

The proteins are synthesized by rabbit reticulocyte lysate with vectors carrying protein sequences of FoxO1, FoxO3, FoxO4 and FoxO6 in 30 incubator for 2 hours. The S³⁵-labeled methionines are added to be incorporated into synthesized proteins during the synthesizing process.

Determination of the Specificity of the FoxO6 Antibody for the FoxO Family

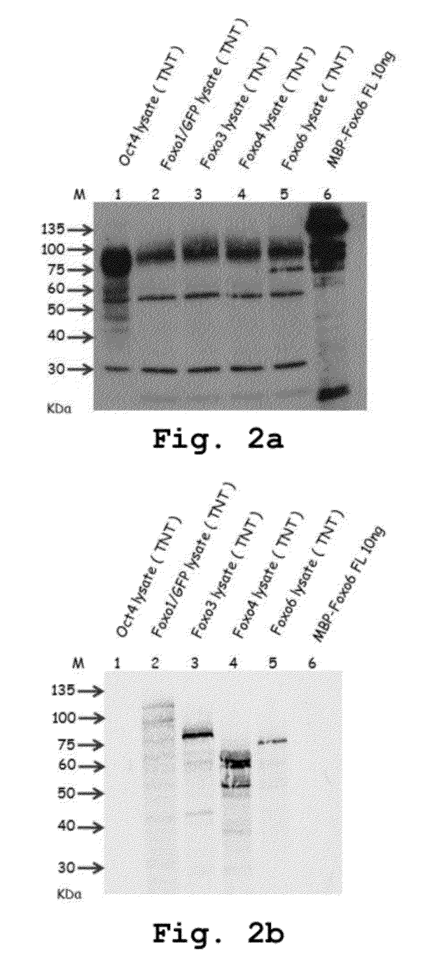

FIG. 2a shows the FoxO1, FoxO3, FoxO4 and FoxO6 proteins generated by in vitro transcription and translation (as mentioned above). The Western blot is performed with the FoxO6 polyclonal antibody (rabbit serum) as the primary antibody (1:10⁵ dilution) and the anti-rabbit-HRP as the secondary antibody (1:10⁴ dilution) (FoxO1/GFP: 99 kDa, FoxO3: 74 kDa, FoxO4: 55 kDa and FoxO6: 62 kDa).

FIG. 2b shows the X-ray film image of PVDF membrane used in FIG. 2a exposed for 1 day, wherein the isotope-labeled protein site of the Fox( ) family could be seen. It is proved that the antibody of the serum could specifically recognize FoxO6 but not other members. The Oct4 TNT lysate is used for the control group of the lysate.

Comparison of the Commercial Antibody with the Antibody of the Present Invention

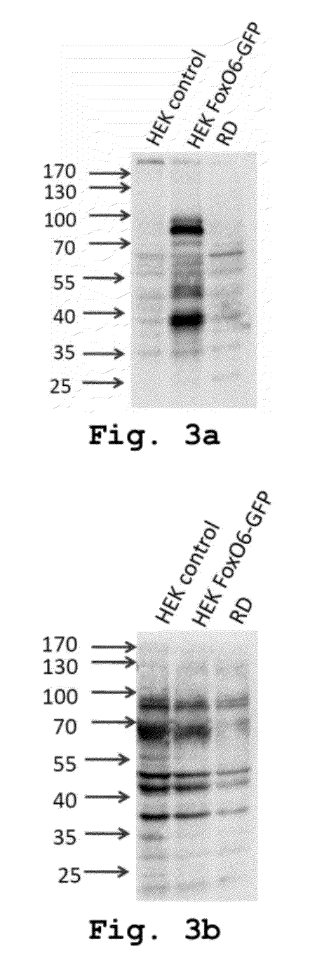

Comparison of specificity: The Western blot is performed with the commercial FoxO6 antibody (Abcam, 1:10³ dilution) or the FoxO6 polyclonal antibody (rabbit serum) as the primary antibody (1:10⁵ dilution) and anti-rabbit-HRP as the secondary antibody (1:10⁴ dilution) to detect the following groups: Non-transfected human embryonic kidney control (HEK control), mFoxO6-transfected overexpressed HEK 293T (HEK FoxO6-GFP) and rhabdomyosarcoma (RD). FIG. 3b shows the result of the commercial antibody recognizing FoxO6. FIG. 3a shows the result of the antibody of the present invention recognizing FoxO6. It is known from FIGS. 3a and 3b that the commercial antibody could not distinguish FoxO6; however, the FoxO6 antibody of the present invention could distinguish FoxO6 (it is known that the size of the human FoxO6 is 491 amino acids which is about 54 kDa).

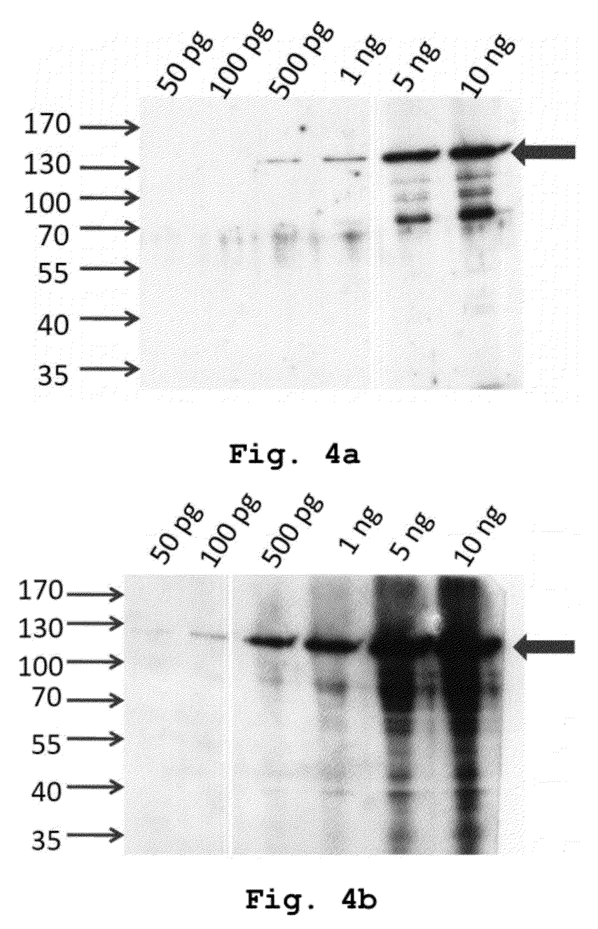

Comparison of the antigen-antibody affinity: using the MBP-FoxO6 full length fusion protein (about 104 KDa) purified from bacteria (BL21) serve as samples, and the 50 pg, 100 pg, 500 pg, 1 ng, 5 ng, and 10 ng of protein were loaded respectively. The Western blot is performed with the commercial FoxO6 antibody (Abcam, 1:10³ dilution) or the FoxO6 polyclonal antibody (rabbit serum, 1:10⁴ dilution) as the primary antibody and anti-rabbit-HRP as the secondary antibody (1:10⁴ dilution) to detect the antigen-antibody affinity. FIG. 4a shows the result of the antigen-antibody affinity by commercial antibody. FIG. 4b shows the result of the antigen-antibody affinity by antibody of the present invention. It is proven that the antigen-antibody affinity by antibody of the present invention is apparently higher than that by the commercial antibody.

### Embodiments

1. A FoxO6 polyclonal antibody, recognizing a FoxO6 sequence having a fragment SEQ ID NO:2 without recognizing any sequence selected from a group consisting of a FoxO1, a FoxO3, a FoxO4 and a combination thereof.
2. The antibody of Embodiment 1, specifically recognizing the FoxO6 sequence.
3. The antibody of any one of Embodiments 1-2, wherein the FoxO6 sequence is a mouse sequence.
4. The antibody of any one of Embodiments 1-3, wherein the FoxO6 sequence is a human sequence.

5. The antibody of any one of Embodiments 1-4, being generated by a mammal.
6. A method for preparing a FoxO6 polyclonal antibody, comprising steps of:
   purifying a FoxO6 sequence having a fragment SEQ ID NO:2;
   mixing the FoxO6 sequence having the fragment SEQ ID NO:2 with an adjuvant to form an antigen; and
   injecting the antigen into a mammal to generate the FoxO6 polyclonal antibody.
7. The method of Embodiment 6, wherein the FoxO6 sequence is a mouse sequence.
8. The method of any one of Embodiments 6-7, wherein the mammal is a rabbit.
9. The method of any one of Embodiments 6-8, wherein the step of purifying is performed by using a microorganism to over-express the FoxO6 sequence.
10. The method of any one of Embodiments 6-9, wherein the step of purifying is performed by a synthesis machine.
11. The method of any one of Embodiments 6-10, wherein the step of injecting comprises a subcutaneous injection.
12. The method of any one of Embodiments 6-11, wherein the FoxO6 polyclonal antibody recognizes a mouse FoxO6.
13. The method of any one of Embodiments 6-12, wherein the FoxO6 polyclonal antibody recognizes a human FoxO6.
14. The method of any one of Embodiments 6-13, wherein the FoxO6 polyclonal antibody does not recognize a sequence being one selected from a group consisting of a FoxO1, a FoxO3, a FoxO4 and a combination thereof.
15. A method for preparing a FoxO6 polyclonal antibody, comprising steps of:
   providing a FoxO6 sequence having a fragment SEQ ID NO:2 as an antigen; and
   injecting the antigen into a mammal to generate the FoxO6 polyclonal antibody.
16. The method of Embodiment 15, wherein the FoxO6 polyclonal antibody specifically recognizes the FoxO6 sequence.
17. The method of any one of Embodiments 15-16, wherein the mammal is a rabbit.
18. The method of any one of Embodiments 15-17, wherein the FoxO6 sequence is purified by using a microorganism to over-express the FoxO6 sequence.
19. The method of any one of Embodiments 15-18, wherein the FoxO6 sequence is purified by a synthesis machine.
20. The method of any one of Embodiments 15-19, wherein the step of injecting comprises a subcutaneous injection.

1. A FoxO6 polyclonal antibody, recognizing a FoxO6 sequence having a fragment SEQ ID NO:2 without recognizing any sequence selected from a group consisting of a FoxO1, a FoxO3, a FoxO4 and a combination thereof.

2. An antibody as claimed in claim 1, specifically recognizing the FoxO6 sequence.

3. An antibody as claimed in claim 1, wherein the FoxO6 sequence is a mouse sequence.

4. An antibody as claimed in claim 1, wherein the FoxO6 sequence is a human sequence.

5. An antibody as claimed in claim 1, being generated by a mammal.

6. A method for preparing a FoxO6 polyclonal antibody, comprising steps of:
purifying a FoxO6 sequence having a fragment SEQ ID NO:1;
mixing the FoxO6 sequence having the fragment SEQ ID NO:1 with an adjuvant to form an antigen; and
injecting the antigen into a mammal to generate the FoxO6 polyclonal antibody.

7. A method as claimed in claim 6, wherein the FoxO6 sequence is a mouse sequence.

8. A method as claimed in claim 6, wherein the mammal is a rabbit.

9. A method as claimed in claim 6, wherein the step of purifying is performed by using a microorganism to over-express the FoxO6 sequence.

10. A method as claimed in claim 6, wherein the step of purifying is performed by a synthesis machine.

11. A method as claimed in claim 6, wherein the step of injecting comprises a subcutaneous injection.

12. A method as claimed in claim 6, wherein the FoxO6 polyclonal antibody recognizes a mouse FoxO6.

13. A method as claimed in claim 6, wherein the FoxO6 polyclonal antibody recognizes a human FoxO6.

14. A method as claimed in claim 6, wherein the FoxO6 polyclonal antibody does not recognize any sequence selected from a group consisting of a FoxO1, a FoxO3, a FoxO4 and a combination thereof.

15. A method for preparing a FoxO6 polyclonal antibody, comprising steps of:
providing a FoxO6 sequence having a fragment SEQ ID NO:1 as an antigen; and
injecting the antigen into a mammal to generate the FoxO6 polyclonal antibody.

16. A method as claimed in claim 15, wherein the FoxO6 polyclonal antibody specifically recognizes the FoxO6 sequence.

17. A method as claimed in claim 15, wherein the mammal is a rabbit.

18. A method as claimed in claim 15, wherein the FoxO6 sequence is purified by using a microorganism to over-express the FoxO6 sequence.

19. A method as claimed in claim 15, wherein the FoxO6 sequence is purified by a synthesis machine.

20. A method as claimed in claim 15, wherein the step of injecting comprises a subcutaneous injection.


SEQUENCE LISTING (1 sequence)

SEQ ID NO: 1 - protein, 264 aa, mouse
KWAASPASHASDDYEAWADFRGSRRPLLGEAAELEDDEALEALAPSSPLMYPSPASALSPALGARCPGELPRLAELGGPLGLHGGGVAGLPDALLDGAQDAYGPRARAGTPSYFGSCKASAYGGGGGFGPPALGSLRRLPMQTIQENKQASFVQAAAPFRPGALPALLPPPPPAPRPGPLLGAPGELALAGAAAAYPGKGAAPYAPPAPSRSALAHPISLMTLPGEAGAAGLAPPAHAAAFGGPPGGLLLDALPGPYAAAAAGP